(19) United States
(12) Patent Application Publication
(10) Pub. No.: US 2023/0248217 A1
(43) Pub. Date: Aug. 10, 2023

(54) ENDOSCOPE FOR LAPAROSCOPIC SURGERY

(71) Applicant: avateramedical GmbH, Jena (DE)

(72) Inventors: Stephan HUNZE, Stadtroda (DE); Eric MARKWEG, Ilmenau (DE); Matthias HÜBNER, Ilmenau (DE)

(21) Appl. No.: 18/165,129

(22) Filed: Feb. 6, 2023

(30) Foreign Application Priority Data
Feb. 7, 2022 (DE) 10 2022 102 804.6

(51) Int. Cl. A61B 1/00; A61B 1/313

(52) U.S. Cl. CPC A61B 1/00096; A61B 1/3132; A61B 1/00193

(57) ABSTRACT

An endoscope for laparoscopic surgery, including a shaft with a central axis, a proximal end, a distal end and a first optical beam path with a prism at the distal end of the shaft and having light entrance and exit surfaces, a lens with an optical axis, the lens behind the prism considered from the distal end of the shaft, and beam deflecting optics behind the lens considered from the distal end of the shaft. The light entrance surface is oriented perpendicular to the central axis, and the light exit surface forms a first angle of inclination with a first spatial axis oriented orthogonal to the central axis. To increase the numerical aperture of the lens, the optical axis is arranged perpendicular to the light exit surface of the prism. As a result, the lens has a cross-sectional area that is larger than the light entrance surface of the prism.

### PRIORITY CLAIM

The present application claims priority to German Patent Application No. 10 2022 102 804.6, filed on Feb. 7, 2022, which said application is incorporated by reference in its entirety herein.

### FIELD OF THE INVENTION

The invention is directed to an endoscope for laparoscopic surgery comprising an endoscope shaft. The endoscope shaft has a central axis, a proximal end, a distal end and a first optical beam path with a prism which is arranged at the distal end of the endoscope shaft and has a light entrance surface and a light exit surface. Behind the prism considered from the distal end of the endoscope shaft, there is arranged a lens with an optical axis. Behind the lens considered from the distal end of the endoscope shaft, there is arranged beam deflecting optics. The light entrance surface of the prism is oriented perpendicular to the central axis, and the light exit surface of the prism forms a predetermined first angle of inclination with a first spatial axis oriented orthogonal to the central axis.

### BACKGROUND OF THE INVENTION

Minimally invasive surgical procedures are used for diverse operations in order to reduce trauma and scarring in the patient. By means of small incisions, entry ports are made for special instruments and an endoscope to which trocars are applied. These trocars are used as guides into the body cavity in which the operation is carried out and seal the incisions. After placement of the trocars, the body cavity is filled with a gas, generally CO2, to create space for the instruments.

Endoscopes are used to allow the surgeon to see into the operating area. Modern endoscopes work digitally and comprise an objective lens, beam deflecting optics and a sensor. A distinction is made between rigid endoscopes and flexible endoscopes.

Rigid endoscopes have a metal shaft and can contain optics which extend over the entire length of the shaft. Images are captured via one or more photodetectors at the proximal end of the shaft. Alternatively, the photodetectors are placed near the distal end so that the optical distance can be kept short.

The resolution of an endoscope is decisively determined by its numerical aperture which depends on the refractive index of the material located between the objective and the sample and on the aperture angle. The aperture angle can in turn be easily influenced by the diameter of the lens. An arrangement of lenses oriented at an angle to the shaft may be advantageous for the resolution. In practice, it is necessary to provide different angles of view so that endoscopes can be used with straight viewing directions as well as with viewing directions which are angled, e.g., by 30°, with respect to the shaft.

An example of such an endoscope is disclosed in U.S. Pat. No. 6,817,975 B1 which suggests both a straight-line endoscope and an angled endoscope. An intermediate image is imaged in an optical element, e.g., a lens. In this way, the intermediate image is imaged in a curved manner in the optical element so that, as a result, a larger numerical aperture is achieved compared to optics in which the intermediate images are located between the optical elements.

An alternative possibility for improving the resolution consists in making the input lens of the endoscope as large as possible. Such a system is disclosed in DE 10 2012 110 905 A1. In this case, the incident light is directed into a prism via a cover glass and a negative lens, twice undergoes total reflection and is coupled into smaller diameter optics. A higher numerical aperture can be achieved and high-resolution sensors installed in this way. However, a construction of this kind only makes sense for angled endoscopes.

### SUMMARY OF THE INVENTION

It is an object of the present invention to provide optics for a straight-line endoscope which has a higher numerical aperture and, therefore, improved resolution.

This object is met in the endoscope described in the introductory part in that the optical axis of the lens is arranged perpendicular to the light exit surface of the prism, and the lens has a cross-sectional area that is larger than the light entrance surface of the prism. This makes it possible to utilize a larger lens with a higher numerical aperture because the angle of inclination allows such a lens to be installed within the confined space of the endoscope shaft.

In an advantageous embodiment, the first angle of inclination is 45°. In this way, the dimension of the lens extending in this spatial direction can be adapted in such a way that a compromise is achieved between maximization of the cross-sectional area and prevention of aberrations. Accordingly, it is possible to increase the diameter of the lens generally. Lens systems or cemented groups can also be used depending on the spatial conditions inside of the endoscope shaft and on the specific design. These lens systems or cemented groups should also fall under the general heading of a lens.

In order to allow the highest-quality imaging, the prism is advantageously formed as a Bauernfeind prism or as a Schmidt prism because these prisms influence imaging only slightly. For the same reason, it is advantageous to form the beam deflecting optics as a parabolic mirror with off-axis beam guiding and/or as a further prism which align/aligns the beam path parallel to the central axis. Accordingly, the light can be received by one or more image sensors which are arranged in an ordered manner and which, as is customary in the art, are disposed perpendicular to the endoscope shaft. This facilitates construction and prevents distortion effects in imaging. It is also possible to orient the image sensors parallel to the central axis and to deflect the light onto the image sensors by means of the beam deflecting optics. The optical setup can be made even more compact in this manner.

In order to further increase the cross-sectional area of the lens, the light exit surface of the prism is preferably oriented in such a way that it forms a predetermined second angle of inclination, preferably 45°, with a second spatial direction oriented orthogonal to the central axis and to the first spatial axis. This makes it possible to achieve the best compromise between maximum numerical aperture and imaging quality for the size of the cross-sectional area of the lens.

The prism preferably comprises two or more partial prisms which are arranged one behind the other, preferably cemented together. In this way, standard components such as Bauernfeind prisms and/or Schmidt prisms can be used for the invention.

In a further advantageous embodiment, the endoscope comprises a second optical beam path which corresponds to the construction of the first optical beam path. The arrangement of the first optical beam path and of the second optical beam path is carried out symmetrically with respect to the central axis. A stereo endoscope based on the concept underlying the invention can be realized in this way. Such a stereo endoscope makes possible an improved display of the surgical site.

In order, on this basis, to further optimize the installation space inside of the shaft of the stereo endoscope, the first optical beam path and the second optical beam path intersect one another or are guided past one another in a particularly preferred embodiment. This makes it possible to provide more space between the optical components so as to facilitate the layout of the individual components and avoid compromises affecting image quality.

It should be understood that the features mentioned above and those yet to be described hereinafter can be used not only in the stated combinations, but also in other combinations or alone, without departing from the scope of the present invention.

### BRIEF DESCRIPTION OF THE DRAWINGS

The invention will be described in more detail in the following based on exemplary embodiments with reference to the accompanying drawings which likewise disclose features key to the invention. These embodiments are to be considered merely as illustrative and not restrictive. For example, it is not to be construed from a description of an embodiment example having a plurality of elements or components that all of these elements or components are necessary to its implementation. On the contrary, other embodiment examples can also contain alternative elements and components, fewer elements or components or additional elements or components. Elements or components of different embodiment examples can be combined unless stated to the contrary. Modifications and variations which are described for one of the embodiment examples may also be applicable to other embodiment examples. In order to avoid repetition, like or comparable elements are designated by like reference numerals in different figures and are not described repeatedly. The drawings show:

FIG. 1 a first construction of an endoscope;

FIG. 2 a distal end of the endoscope;

FIG. 3 a schematic view of a second construction of an endoscope;

FIGS. 4A, 4B diagrams illustrating the principle of enlargement of a lens diameter.

### DETAILED DESCRIPTION

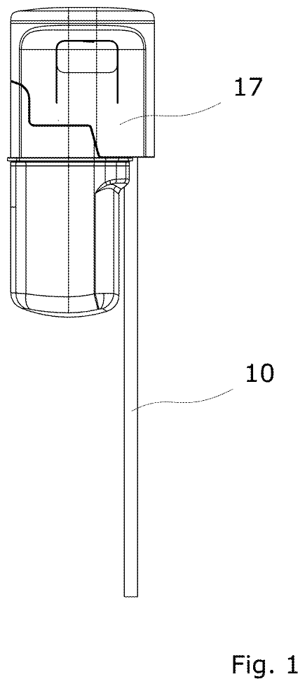

FIG. 1 shows an endoscope with an endoscope shaft 10 and an endoscope housing 17. The optical components are located inside of the endoscope shaft 10. In a standard rod endoscope, the incident light impinges on input optics at a distal end of the endoscope shaft 10, is shaped via various optical elements and is subsequently guided by means of rod optics to the distal end of the endoscope shaft 10 at which the endoscope housing 17 is located. The image sensors are mostly arranged in this endoscope housing 17. Alternatively, in so-called chip-on-tip endoscopes, the image sensors can be arranged in the region of the distal end of the endoscope shaft 10. In this type of endoscope, the incident light is likewise shaped through corresponding optical components. In chip-on-tip endoscopes, however, the rod optics are dispensed with because the light impinges directly on sensors arranged at the distal end of the endoscope shaft 10. Stereo endoscopes which have two separate image channels are usually used in surgery.

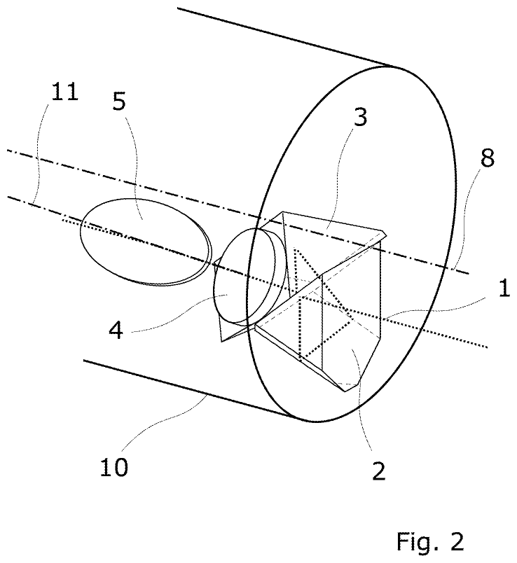

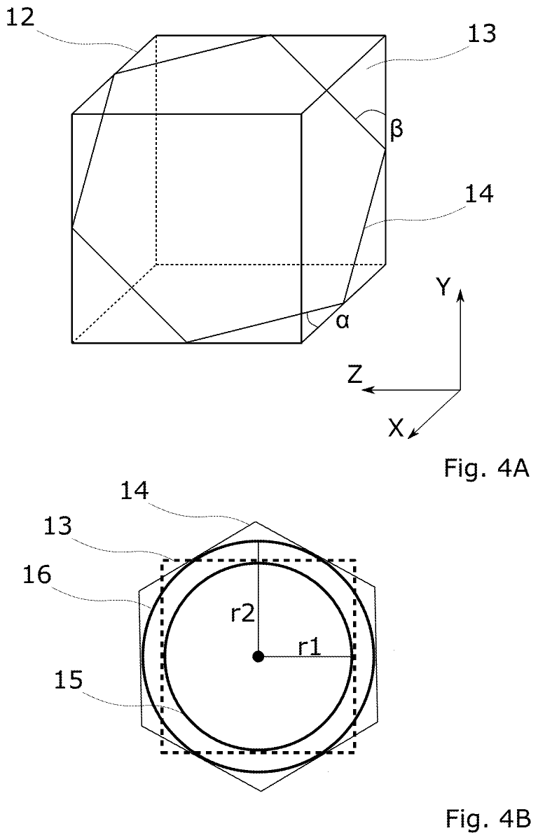

FIG. 2 shows the distal and of an endoscope, only one of the two image channels being shown here. A light beam 1 impinges perpendicularly on a prism which comprises a first partial prism 2 and a second partial prism 3. The light beam 1 runs parallel to a central axis 8 of the endoscope extending centrally through an endoscope shaft 10 and impinges on a light entrance surface of the first partial prism 2 which is oriented perpendicularly relative to the central axis 8 and, therefore, also relative to the light beam 1. In the present embodiment form, the prism is a cemented group comprising the first partial prism 2 which is constructed as a Bauernfeind prism and the second partial prism 3 in the form of a Schmidt prism. However, a one-piece construction is also easily possible. The two partial prisms 2, 3 deflect the light beam 1 repeatedly and, accordingly, in two spatial planes at a first prism angle and a second prism angle. At a light exit surface located on the side of the second partial prism 3 remote of the first partial prism 2, the light beam 1 again exits the second partial prism 3. The light exit surface of the second partial prism 3 forms a predetermined first angle of inclination with a first spatial axis oriented orthogonal to the central axis 8. Further, the light exit surface forms a predetermined second angle of inclination with a second spatial axis oriented orthogonal to the central axis 8 and to the first spatial axis. This arrangement will be discussed in detail later referring to FIGS. 4A and 4B. Arranged parallel to the light exit surface is a lens 4 with an optical axis 11 extending perpendicular to the light exit surface and has a larger cross-sectional area than the light entrance surface. The light beam 1 subsequently impinges on a parabolic mirror 5 as beam deflecting optics. This can be an off-axis parabolic mirror. The light beam 1 is now again oriented parallel to the central axis 8 by means of this off-axis parabolic mirror and is guided to a sensor, not shown here, or an eyepiece at a proximal end of the endoscope shaft 10. A parabolic mirror 5 has the advantage that, apart from deflecting the light beam 1, it also has beam-shaping characteristics in order to adapt the light beam 1 to the subsequent optical elements such as light-conducting elements and/or sensors. Instead of the parabolic mirror 5, a further prism can also be used in order to achieve the desired beam deflection. Together with the optional light-guiding optics, not shown here, and the sensor, the construction described here comprising prism, lens 4 and parabolic mirror 5 forms a first optical beam path.

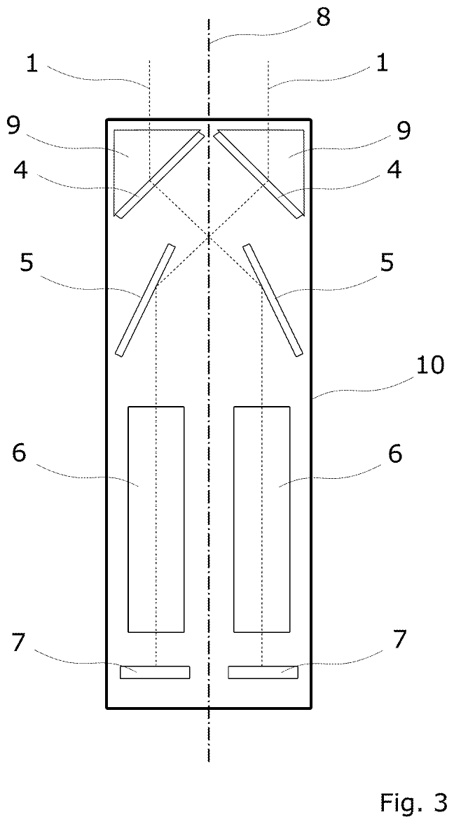

When the beam path presented in FIG. 2 is introduced twice in an endoscope shaft 10, a stereo endoscope with two image channels can be realized. Such a stereo endoscope is shown in a schematic sectional view in FIG. 3 in which the endoscope comprises a second optical beam path which corresponds to the construction of the first optical beam path. The arrangement of the first optical beam path and second optical beam path is carried out rotationally symmetric to the central axis 8.

The incident light beams 1 impinge on the light entrance surfaces of the prisms 9 and are deflected in direction of at least a first angle of inclination. In this embodiment example, every prism 9 is formed in one piece. After passing through the lens 4, the light beams 1 in this embodiment example are guided in direction of the central axis 8 in which the light beams 1 intersect or are guided past one another. A construction of this kind facilitates the layout of the optical elements which are used in spite of the limited installation space inside of the endoscope shaft 10. Accordingly, for example, the radius of curvature of the lens 4 can be selected to be larger, which reduces imaging errors. When the light beams 1 intersect, interference can generally result insofar as the light is coherent light. Since both prisms 9 capture the same scene from different perspectives to achieve a stereo effect, this plays a subordinate role for the captured image; only the illumination light, which is usually coherent, can be affected by this. However, since this illumination light impinges on the light entrance surfaces of the prisms 9 in a disorganized manner, coupled-in scatter light can be eliminated by shutters at the focus point of the respective lens 4. If interference effects occur nevertheless, they only slightly influence the image quality and, if necessary, can be corrected within the framework of electronic image processing. If the light beams 1 are guided past one another, interference can no longer occur.

After passing the central axis 8, the light beams 1 also impinge on the parabolic mirror 5 in this embodiment example, are aligned at the latter parallel to the central axis 8 and are guided, in each instance, to relay optics 6 which in turn direct the light to the proximal end of the endoscope shaft 10 to sensors 7.

As has already been mentioned, the lens 4 has a larger cross-sectional area than the light entrance surface. The beam path between lens 4 and the object to be captured is deflected repeatedly in the prism 9 and accordingly lengthened. As a result of the inclined arrangement inside of the endoscope by the first and second angles of inclination, the lens 4 can turn out larger than would be the case if it were arranged parallel to the light entrance surface so that the numerical aperture is increased. This will be described in the following referring to FIGS. 4A and 4B.

For the sake of simplicity, the possible space in which a lens 4 can be accommodated is shown in FIG. 4A as a cube 12 which has an entrance surface 13. The spatial axes are defined with reference to the coordinate system (X, Y, Z) shown in the drawing. The light comes from incident direction Z and impinges perpendicularly on the entrance surface 13. A circular lens 4 which is located inside of the entrance surface 13 can have, at most, a diameter corresponding to the side length of the cube 12. However, in three-dimensional space it is possible to introduce a larger lens 4 which is completely covered by the entrance surface 13 considered from the incident direction Z. To this end, the lens 4 is to be arranged in a spatial plane which forms a first angle of inclination α with a first spatial axis X and a second angle of inclination R with a second spatial axis Y. The first spatial axis X and the second spatial axis Y extend perpendicular to the incident direction Z and are likewise oriented perpendicular to one another. Both angles of inclination a, R are preferably 45° because the attainable circular area, and therefore the possible cross-sectional area of the circular lens 4, reaches a maximum there.

With a perpendicular incidence of light so that the lens lies completely within the entrance surface 13, the lens 4 can have a maximum diameter which corresponds to the side length of the cube 12. In other words, the cross-sectional area of such a lens 4 is described by a first circle 15 which has a first radius r1 corresponding to one half of the side length of the cube 12. The surface area of the first circle 15 is calculated as π*r1².

The entrance surface 13 lies in a plane which is defined by the first spatial direction X and second spatial direction Y and is oriented perpendicular to the incident direction Z. When this plane is tilted by the first angle of inclination α and by the second angle of inclination β, in this case by 45° in each instance, the cross-sectional area located in the plane inclined by the angles of inclination α, β and enclosed by the cube 12 is a hexagon 14. From geometrical considerations, it can be deduced that a second circle 16 located entirely inside of the hexagon 14 has a second radius r2 which is increased over the first radius r1 by a factor of \sqrt{3/2}. This corresponds to a 50% increase in the cross-sectional area. Compared with entrance surface 13 of cube 12, which corresponds to the entrance surface of prism 9, the cross-sectional area of such a lens 4 which corresponds to that of the second circle 16 is still increased by a factor of (3 π/8). Since the numerical aperture is directly proportional to the diameter of the optics in question, the numerical aperture can also be correspondingly increased in this way.

### REFERENCE CHARACTERS

1 light beam
2 first partial prism
3 second partial prism
4 lens
5 parabolic mirror
6 relay optics
7 sensor
8 central axis
9 prism
10 endoscope shaft
11 optical axis
12 cube
13 entrance surface
14 hexagon
15 first circle
16 second circle
17 endoscope housing
X first spatial axis
Y second spatial axis
Z incident direction
r1 first radius
r2 second radius
α first angle of inclination
β second angle of inclination

What is claimed is:

1. An endoscope for laparoscopic surgery, comprising:
an endoscope shaft having a central axis, a proximal end, a distal end and a first optical beam path, and including:
a prism arranged at the distal end of the endoscope shaft and having a light entrance surface and a light exit surface,
a lens with an optical axis, the lens being arranged behind the prism as considered from the distal end of the endoscope shaft, and
beam deflecting optics arranged behind the lens as considered from the distal end of the endoscope shaft, and
wherein the light entrance surface of the prism is oriented perpendicular to the central axis, and the light exit surface of the prism forms a predetermined first angle of inclination with a first spatial axis oriented orthogonal to the central axis, and the optical axis of the lens is arranged perpendicular to the light exit surface of the prism, and the lens has a cross-sectional area that is larger than the light entrance surface of the prism.

2. The endoscope according to claim 1, wherein the first angle of inclination is 45°.

3. The endoscope according to claim 1, wherein the prism is formed as a Bauernfeind prism or as a Schmidt prism.

4. The endoscope according to claim 2, wherein the prism is formed as a Bauernfeind prism or as a Schmidt prism.

5. The endoscope according to claim 1, wherein the beam deflecting optics comprise a parabolic mirror with off-axis beam guiding and/or a further prism which align/aligns the beam path parallel to the central axis.

6. The endoscope according to claim 2, wherein the beam deflecting optics comprise a parabolic mirror with off-axis beam guiding and/or a further prism which align/aligns the beam path parallel to the central axis.

7. The endoscope according to claim 3, wherein the beam deflecting optics comprise a parabolic mirror with off-axis beam guiding and/or a further prism which align/aligns the beam path parallel to the central axis.

8. The endoscope according to claim 1, wherein the light exit surface of the prism forms a predetermined second angle of inclination, preferably 45°, with a second spatial axis oriented orthogonal to the central axis and to the first spatial axis.

9. The endoscope according to claim 8, wherein the predetermined second angle of inclination is 45°.

10. The endoscope according to claim 1, wherein the prism comprises two or more partial prisms which are arranged one behind the other.

11. The endoscope according to claim 10, wherein the two or more partial prims are cemented together.

12. The endoscope according to claim 1, wherein the endoscope comprises a second optical beam path which corresponds to a construction of the first optical beam path, and the arrangement of the first optical beam path and of the second optical beam path is carried out symmetrically with respect to the central.

13. The endoscope according to claim 12, wherein the first optical beam path and the second optical beam path intersect one another.

14. The endoscope of claim 12, wherein the first optical beam path and the second optical beam path are guided past one another.
